(19) United States
(12) Patent Application Publication
(10) Pub. No.: US 2009/0221056 A1
(43) Pub. Date: Sep. 3, 2009

(54) Pathogen-Detecting Cell Preservation Systems

(76) Inventors: Martha S. Petrovick, Barre, MA (US); Eric D. Schwoebel, Woburn, MA (US); Frances E. Nargi, Littleton, MA (US); James D. Harper, Jamaica Plain, MA (US); Todd H. Rider, Littleton, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/085,486

(22) PCT Filed: Nov. 30, 2006
(86) PCT No.: PCT/US2006/045825

§ 371 Date: Feb. 6, 2009

Related U.S. Application Data
(60) Provisional application No. 60/741,384, filed on Nov. 30, 2005.

(51) Int. Cl. C12N 1/12

(52) U.S. Cl. 435/257.6

(57) ABSTRACT

Methods to produce cells that remain viable at ambient or non-refrigerated temperatures, or which can be stored in a dry state are described. In particular, cell preservation is for long term storage of cells, such as mammalian cells, at ambient or non-refrigerated temperatures, while retaining cell viability. Also provided are sensor cells that can detect target particles, biological agents or other materials and which remain viable at ambient or non-refrigerated temperatures, or which can be stored in a dry state.

### RELATED APPLICATION

This application claims the benefit of U.S. Provisional Application No. 60/741,384, filed on Nov. 30, 2005.

The entire teachings of the above application are incorporated herein by reference.

### GOVERNMENT SUPPORT

The invention was supported, in whole or in part, by Government funds from U.S. Air Force contract number F19628-00-C-0002. The Government has certain rights in the invention.

### BACKGROUND OF THE INVENTION

The need for small, fast, and sensitive detectors of biological agents which are able to monitor an environment for extended periods of time is underscored by the proliferation of biological and chemical weapons. Under battlefield conditions, a useful detector would rapidly alert a soldier when a specific biological or chemical agent is detected so that countermeasures can quickly be implemented. Such detectors would be useful in non-military applications as well. Rapid detection of antibiotic-resistant bacteria in a patient would help clinicians select a more effective therapeutic regimen. Continuous monitoring of a city's drinking water supply would provide early warning of potential pathogens, giving public works officials more time to manage the potential health risks to the public. In addition, the use of these detectors in meat and poultry inspections would be a significant improvement over the current “poke-and-smell” procedure. In general, such detectors are sorely needed analytical and diagnostic applications within the fields of medicine (e.g., veterinary medicine), agriculture, environmental protection (e.g., to diagnose sick building syndrome), and food processing or regulation.

Devices that exploit antibody diversity for detection of multiple and rare target particles or antigens have been described in, for example, U.S. Pat. No. 6,087,114 and U.S. Pat. No. 6,248,542. These devices generally include a liquid medium containing sensor cells (e.g., a B cell, macrophage or fibroblast), also referred to herein as “CANARY” cells or “emitter” cells, an optical detector, and liquid medium receiving target particles to be detected. Although mammalian cells can be stored frozen at temperatures of minus 80° C. or colder, it is well known that they lose viability within weeks when stored in a liquid state at refrigerated or at ambient temperatures. Furthermore, the technology to store cells in a dry state for extended periods while maintaining viability remains under development. Storage of sensor cells at temperatures and conditions other than minus 80° C. or colder would greatly improve the convenience of devices that use sensor cells.

Therefore, a need exists for cells, such as mammalian cells that remain viable at ambient or non-refrigerated temperatures, or which can be stored in a dry state. In particular, a need exists for sensor cells that can detect biological agents or other materials and which remain viable at ambient or non-refrigerated temperatures, or which can be stored in a dry state.

### SUMMARY OF THE INVENTION

The present invention provides methods for preserving cell viability when cells are stored at ambient or non-refrigerated temperatures, which would otherwise result in reduced or lost cell viability.

In one embodiment, the cell preservation technique is for an emittor cell (also referred to herein as a CANARY cell, or a sensor cell). In a particular embodiment, emitter cell expresses one or more receptors for a target particle. In one embodiment, the receptor is an antibody. In another embodiment, the receptor is an Fc receptor. In a further embodiment, the emittor cell is a B cell, a macrophage or a fibroblast cell.

In a still further embodiment, the emitter cell expresses an emittor molecule. In a particular embodiment, the emittor molecule emits a photon in response to an increase in intracellular calcium. In a further embodiment, the emittor molecule is aequorin.

In a further embodiment, the method for cell preservation comprises protecting the cell (such as an emittor cell) from apoptosis, oxidation, and/or protein degradation with combinations of one or more caspase inhibitors, protease inhibitors, and/or proteosome inhibitors.

In another embodiment, the method for cell preservation comprises additionally or alternatively, increasing the expression of genes in the cell that are responsible for inducing and/or maintaining quiescence, thereby increasing the time the cells can remain in a quiescent state.

In a further embodiment, the method for cell preservation comprises additionally or alternatively controlling expression of genes that regulate apoptosis. In a particular embodiment, an Inhibitor of Apoptosis Protein (IAP) is expressed in the cell.

In another embodiment, the method for cell preservation comprises additionally or alternatively, extending the conditions to include long-term storage in liquid at ambient and refrigerated temperatures.

In a further embodiment, the method for cell preservation comprises additionally or alternatively, inducing a protective quiescent state comprising the addition of cell-cycle inhibitors. In a particular embodiment, cell cycle inhibitors are selected from the group consisting of actinomycin D, mitomycin C, rapamycin, mevastatin, tunicamycin and wortmannin.

The invention described herein also provides for an emittor cell that is an extremophile. In a particular embodiment, the extremophile is a desiccation-tolerant extremophile. In one embodiment, the extremophile is a chlamydomonas algae. In a particular embodiment, the extremophile expresses one or more receptors for a target particle. In one embodiment, the receptor is an antibody. In another embodiment, the receptor is an Fc receptor.

In a further embodiment, the extremophile expresses an emittor molecule. In a particular embodiment, the emittor molecule emits a photon in response to an increase in intracellular calcium. In a further embodiment, the emittor molecule is aequorin.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing and other objects, features and advantages of the invention will be apparent from the following more particular description of preferred embodiments of the invention, as illustrated in the accompanying drawings. The drawings are not necessarily to scale, emphasis instead being placed upon illustrating the principles of the invention.

FIG. 1 is an outline of cell preservation techniques and a photograph of B cells during spheroid formation.

FIG. 2 is a graph demonstrating activity of cells following one week storage at room temperature.

FIG. 3 is a line graph demonstrating activity of cells following one week storage at room temperature and a bar graph of the percent of viable cells following 1-3 weeks storage at room temperature, with or without a Pan-Caspase III inhibitor.

FIG. 4 is a graph demonstrating the results of a gene expression analysis during induction of quiescence, storage and revival of HEK293 cells.

FIG. 5 is an overview of B-cell logistics of the conditions of storage and use of cells at different temperatures and under dry storage conditions.

FIGS. 6(a) and 6(b) are graphs of results with treatments and additives that enhance the activity of CANARY cells stored long term. FIG. 6(a) is a graph demonstrating improved viability and activity of cells stored at 4° C. when the cells had been incubated with wortmannin prior to preparation for the assay, and were stored in the presence of the caspase inhibitor Z-LEED-FMK and the antioxidants N-acetylcysteine, sodium selenite, deferoxamine, and aminoguanadine. FIG. 6 (b) is a graph demonstrating improved viability and activity of cells stored at room temperature when the cells had been incubated with tunicamycin prior to preparation for the assay, and were stored in the presence of Caspase Inhibitor III. This treatment extended the length of time the cells could be stored at room temperature without loss of activity from 2 days to 1 week.

FIG. 7 is a graph of the expression pattern (levels) of genes in CANARY cells after 24 hr treatment with 2% DMSO (“1” on the x-axis), after 24 hr rotation at room temperature (“2” on the x-axis), and after being stored for 1 week at 4° C. (“3” on the x-axis), as compared to control (“0” on the x-axis).

FIG. 8 is a graph demonstrating that overexpression of the Artemia franciscana heat-shock gene, artemin, improves the activity of cells stored at room temperature, increasing the storage time to 1 week without loss of activity.

FIG. 9 is a bar chart demonstrating that overexpression of the anti-apoptotic gene, Bcl-XL, improves both the viability and activity of cells stored at 4° C., increasing the storage time from 2 to 4 weeks.

FIG. 10 is a graph demonstrating a comparison of the activity of freshly prepared vs. stored CANARY cells. Equal numbers (1600 cells) of freshly prepared (Fresh) cells specific for Y. pestis, and those that had been stored at room temperature for 3 weeks (Stored) were compared for their response to antigen in a CANARY assay.

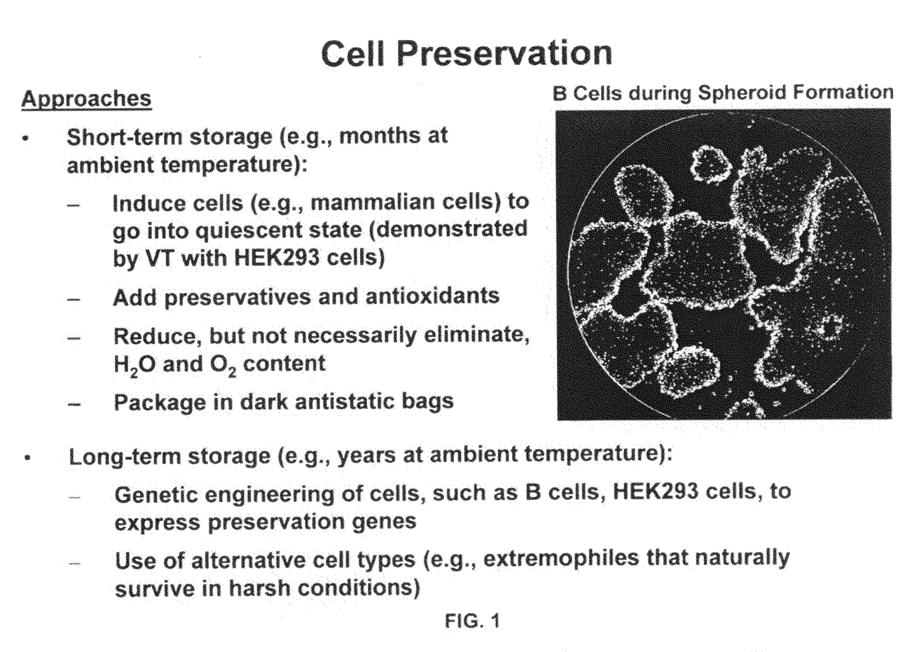

### DETAILED DESCRIPTION OF THE INVENTION

Although mammalian cells can be stored frozen at temperatures of −80° C. or lower, it is well known that they lose viability within several weeks when stored in a liquid state refrigerated or at ambient temperatures. Furthermore, the technology to store cells in a dry state for extended periods while maintaining viability remains under development. Several approaches to improve dry storage of viable cells have involved the use of non-reducing disaccharides such as trehalose, either intra- or extracellular, but these have enjoyed limited success [Nat. Biotechnol., 2000, 18(2): 168-171; FEBS Lett., 2000, 487: 199-202; J. Opthalmol., 85: 610-612 (2001); Cryobiology, 42(3): 207-217 (2001); J. Physiol., 558: 181-191 (2004)]. The six-week ambient-temperature storage of a human cell line was recently achieved by inducing a quiescent state, while maintaining some level of water content and reducing available oxygen and static electricity [Jack et al., J. Cell. Physiol., 206(2): 526-536 (2005)]. The quiescent state was induced by growing the cells in three-dimensional structures termed spheroids, and is believed to provide protection from apoptosis and other forms of cell death.

The present invention improves on this technology using one or more of the following cell preservation techniques comprising: (1) providing further protection from apoptosis, oxidation, and/or protein degradation with combinations of caspase inhibitors (for example, Z-AEVD-FMK, Z-DEVD-FMK, Z-LEED-FMK, Z-LEHD-FMK, Z-WEHD-FMK, Z-VAD-FMK, Z-VDVAD-FMK, Z-YVAD-FMK, Z-VEID-FMK, Z-IETD-FMK, OPH-109, and/or Pan-Caspase III inhibitor), antioxidants (for example, sodium selenite, N-acetyl-cysteine, deferoxamine, aminoguanidine, Trolox, ebselen, and/or Tempol), and/or protease and/or proteosome inhibitors (for example, AEBSF, aprotonin, bestatin, calpeptin, cathepsin L inhibitor, E-64, leupeptin, and/or pepstatin A); (2) increasing the expression of genes that are responsible for inducing and maintaining quiescence in order to increase the time cells can remain in a quiescent state (such genes can be identified, for example, in a transcriptional analysis using techniques that are known to those of skill in the art). Increased gene expression can be achieved by any means known in the art, for example, by overexpressing the gene or genes in the cell with a strong promoter; (3) controlling expression of genes that regulate apoptosis, for example, by genetic methods, such as overexpression of an Inhibitor of Apoptosis Protein (IAP) such as XIAP; (4) extending the conditions to include long-term storage in liquid at ambient and refrigerated temperatures; and/or (5) inducing a protective quiescent state with the use of cell-cycle inhibitors (for example, actinomycin D, mitomycin C, rapamycin, mevastatin, tunicamycin, and/or wortmannin). As used herein, “long-term storage” refers to periods of time greater than about 10 years, greater than about 1 year, greater than about 9 months, greater than about 6 months, greater than about 3 months, greater than about 2 months, greater than about 1 month, greater than about 2 weeks, greater than about 1 week, or greater than about 72 hours.

Furthermore, as described herein, the present invention provides methods for cell preservation, such as an emittor cell, in liquid, semi-liquid, or desiccated states at ambient, refrigerated or non-refrigerated temperatures. As used herein, ambient or non-refrigerated temperatures range from about 5° C. to about 50° C., from about 10° C. to about 40° C., from about 15° C. to about 30° C., and from about 20° C. to about 30° C.

In a particular embodiment, one or more of the cell preservation techniques is applied to an emittor cell (also referred to herein as a CANARY cell, or a sensor cell). As used herein, an emittor cell is used for the detection of a target particle (such as a biological antigen, a soluble antigen, a nucleic acid, a toxin, a chemical, and the like). Detection of the target particle is mediated in part by binding of the target particle to a receptor, either directly or indirectly, expressed on the cell surface of the emittor cell. Direct binding can be via a receptor, such as an antibody, which binds directly and specifically to the target particle. Indirect binding of the target particle can be through, e.g., an Fc receptor that binds to an antibody that is attached (e.g., bound) to the target particle. Binding of the antigen to the receptor results in an increase in calcium concentration. The emitter cells also contain one or more emitter molecules (e.g., aequorin) in their cytosol, which emit photons in response to the increased calcium concentration in the cytosol. The photon emission can be detected, thereby detecting the presence of the target particle. This is also referred to herein as a “CANARY assay”. For further information on emittor cells, optoelectronic detection systems using same, and CANARY assays, see for example, U.S. patent application Ser. No. 11/001,583, U.S. Patent Application No. 2004/0121402, U.S. Pat. No. 6,087,114, and U.S. Pat. No. 6,248,542, the teachings of all of which are incorporated herein by reference in their entirety.

An alternative approach is the genetic modification of desiccation-tolerant extremophiles for use as a CANARY cell. For example, chlamydomonas algae are known to form spores when their local environmental habitat dries up from lack of rainfall. These spores have been reported to re-hydrate to viability over 70 years after sporulation. Further, chlamydomonas is known to have a cell-surface-receptor-activated calcium-signaling transduction cascade similar to that in B cells. Therefore, an extremophile can be genetically engineered to express one or more receptors, such as an antibody or an Fc receptor, and an emittor molecule, such as aequorin. The expression of the receptor on the cell surface when crosslinked by a pathogen or antigen stimulates a signaling cascade causing an increase in intracellular calcium concentration, and this increased concentration of intracellular calcium ions stimulates the emittor molecule to emit light which can be measured and correlated with the presence of the target antigen or pathogen. Thus, an extremeophile emittor cell can be stored for extended periods of time (e.g., long-term storage) in a dehydrated state, e.g., at ambient, refrigerated or non-refrigerated temperatures, and remain viable upon re-hydration for use in, e.g., a CANARY assay.

### EXEMPLIFICATION

### Example 1

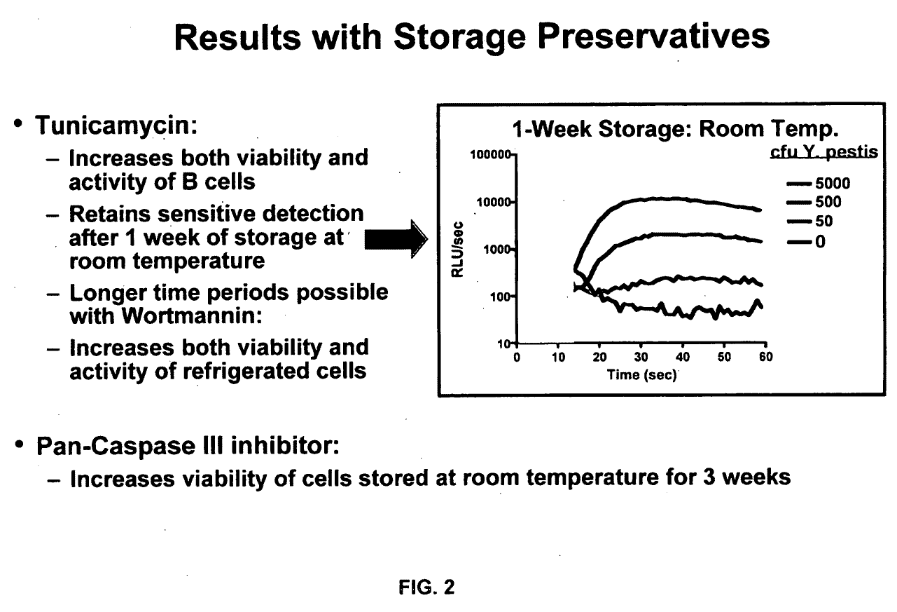

Tunicamycin increases both cell viability and activity of CANARY B cells (see FIG. 2). Following storage of CANARY B cells (expressing a receptor specific for Yersinia pestis) with tunicamycin at room temperature, the CANARY B cells retained detection sensitivity for Yersinia pestis (see graph, FIG. 2).

Furthermore, treatment of CANARY cells with wortmannin also increased both viability and antigen-detection activity of refrigerated CANARY cells.

### Example 2

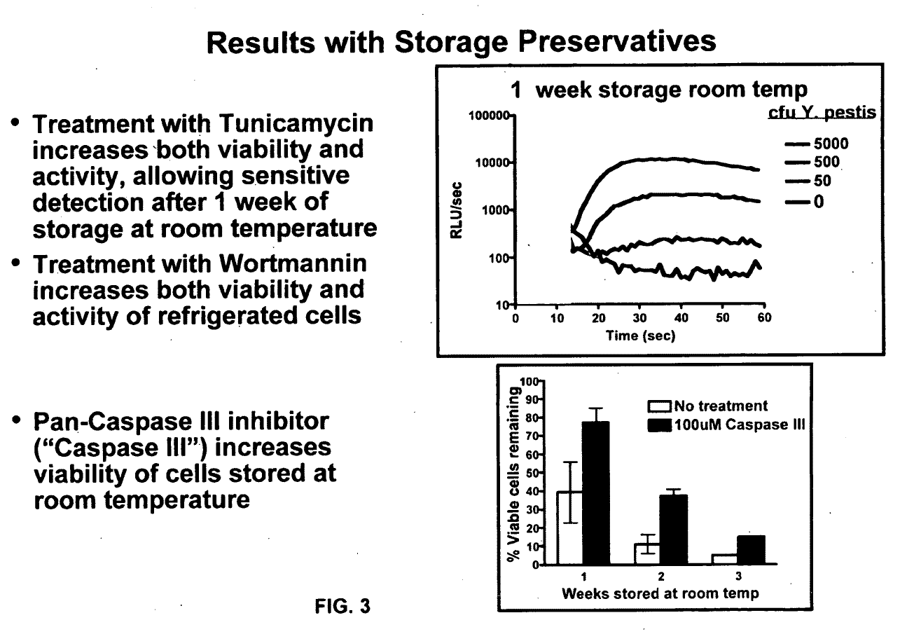

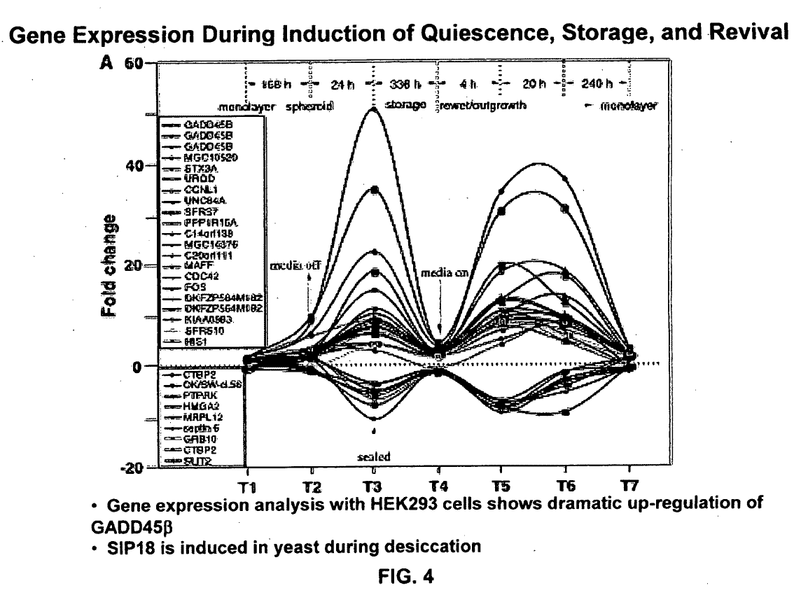

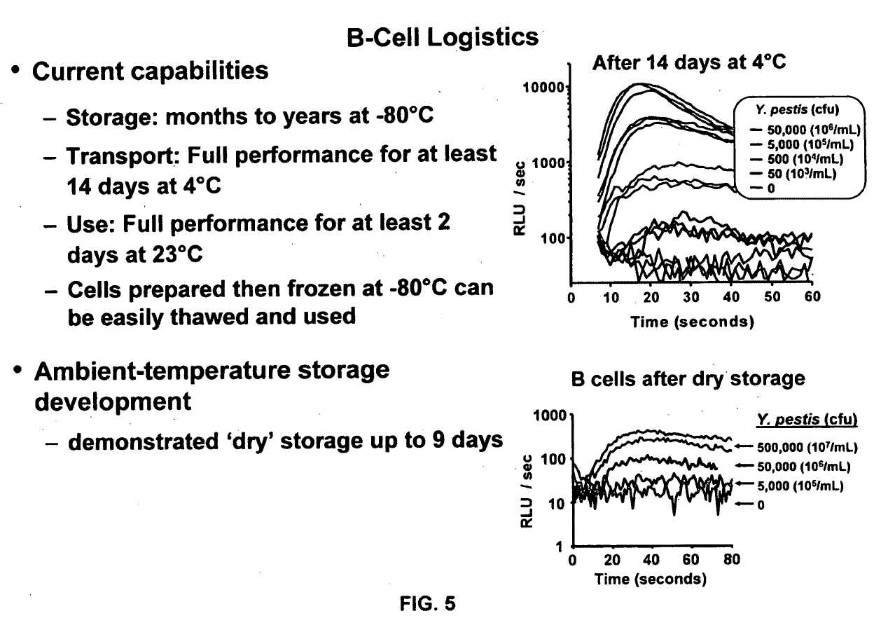

Treatment of cells with Pan-Caspase III inhibitor (referred to as “Caspase III” in FIG. 3, bar chart) increases viability of cells stored at room temperature for one, two and three weeks, as compared to cells stored at room temperature for one, two and three weeks without wortmannin (see FIG. 3, bar chart).

### Example 3

### Demonstrated Improvements in Cell-Storage Logistics

The CANARY B-cell bioagent sensor technology demonstrates the best combination of speed and sensitivity for any bioagent-identification technology known [Rider et al., Science 301: 213 (2003)]. CANARY can detect pathogens in many matrices and formats including air, food, surfaces, and medical samples. However, there is still a need for the easy and inexpensive storage and logistics of the B cells themselves. The cells are typically kept frozen or refrigerated until ready to use, which is acceptable (though non-optimal) for many medical and homeland environments, but is not optimal for all circumstances, e.g., for forward-deployed military units. In addition, most users desire an emittor cell (e.g., a B-cell) reagent kit that can sit on a shelf in a warehouse or laboratory at ambient temperature for periods from about 6 months to up to about 10 years or more and that can be taken out, loaded into a sensor, and used for a test. Such kits would comprise e.g., the emittor cell and optionally instructions for using the emittor cells in an assay, such as a CANARY assay.

Prior to Applicants' instant invention, the shelf life of the CANARY cell reagent was 2 days at room temperature and 2 weeks at 4° C. The cells can be stored frozen indefinitely at temperatures of −80° C. or less, but this requires liquid nitrogen or special freezers that not all laboratories may have. Described herein are examples of a variety of treatments, additives, and overexpression of genes to improve both the viability and the activity of cells stored for long term at room temperature (e.g., ambient temperature) and/or 4° C.

### Methods and Materials

The chemical additives investigated were: caspase inhibitors AEVD, DEVD, LEED, LEHD (toxic at 50 μM, WEHD (toxic at 50 μM), VAD, VDVAD, YVAD, VEID, IETD (R&D Systems), OPH-109 (MP Biomedicals), Caspase-Inhibitor III (Calbiochem); protease inhibitors AEBSF, aprotonin, bestatin, calpeptin, cathepsin L inhibitor, E-64, leupeptin, pepstatin A (Sigma); cell-cycle inhibitors rapamycin (LC Labs), actinomycin D, mitomycin C, mevastatin, tunicamycin, wortmannin (Sigma); antioxidants sodium selenite, N-acetyl-cysteine, deferoxamine, aminoguanidine, Trolox, ebselen, Tempol (Sigma), MnTBAP (A.G. Scientific).

For storage at 4° C., cells were incubated at a density of 2.5-3×10⁵ cells/mL in 1-3-μM wortmannin for 24 h at 37° C., then incubated in 2% DMSO at a concentration of 5×10⁵ cells/mL for 24 h at 37° C. Then cells were incubated in the dark at room temperature for 2 h in assay medium [CO₂-Independent medium, 10% fetal bovine serum, 50-μg/ml streptomycin, 50-U/ml penicillin, and 250 ng/mL amphotericin B (Life Technologies)] with 50-μM coelenterazine (Molecular Probes, Eugene, Oreg.). The cells were then washed twice, resuspended at a final concentration of 5×10⁵ cells/mL in assay medium with the following additions: 100-μM Z-LEED-FMK, 50 μg/mL N-acetylcysteine, 150 ng/mL sodium selenite, 15 μM deferoxamine (freshly made stock), 1.5 mM aminoguanadine (freshly made stock), and left to rotate overnight at room temperature.

For storage at room temperature cells were incubated at a density of 2.5-3×10⁵ cells/mL in 0.1 μg/mL tunicamycin for 24 h at 37° C., then incubated in 2% DMSO at a concentration of 5×10⁵ cells/mL for 24 h at 37° C. Then cells were incubated in the dark at room temperature for 2 h in assay medium with 50-μM coelenterazine, washed twice and resuspended at a final concentration of 5×10⁵ cells/mL in assay medium with 100-μM Caspase Inhibitor III.

Tubes of cells were vacuum-sealed in Food Saver bags using a MagicVac sealer, and head space was provided by storing cells in 15-mL and 50-mL tubes that were not filled to capacity. Cells stimulated with anti-CD40 were incubated with the antibody (BD Biosciences) at a concentration of 2×10⁵ cells/mL for 24 h at 37° C., then treated with 2% DMSO and incubated in coelenterazine as described above. Heat-shock conditions were 42° C. for 2-5 h. The heat-shock response was measured by transfecting with an Hsp70-responsive promoter driving the expression of firefly luciferase and a renilla luciferase plasmid as a control for transfection efficiency. Cells were transfected by electroporation, allowed to recover for 24-48 h, subjected to heat shock, and assayed the next day with the Dual Luciferase Reporter Assay (Promega).

Cells transfected with Artemia p26 and artemin in pcDNA3.1 plasmids were selected in 500-μg/mL Hygromycin (Invitrogen). Murine GADD45β was cloned by RT-PCR and inserted in pEF6/V5/His-TOPO (Invitrogen). Cells transfected with this plasmid were selected in 5-μg/mL Blasticidin (Invitrogen). Restriction sites were added to the human Bcl-XL gene (Invivogen) by PCR, it was inserted into the NheI and XhoI sites of pcDNA3.1 (Invitrogen) and the sequence confirmed. Other genes that were overexpressed but conferred no protection during storage include: Artemia p22 and p21 (two versions, one with G at amino acid position 62 and one with E), BiP, XIAP, baculovirus p35, and a deletion mutant of HSF1 (amino acids 221-315) that has been shown to be constitutively active [Voellmy, Methods 35: 199 (2005)].

Cells were transfected by electroporation (BioRad) at 270 V, 950 μF and cloned by limiting dilution. Clones were analyzed for expression by quantitative RT-PCR using TaqMan® Gene Expression Assays (Applied Biosystems). Several clones with moderate and high expression were chosen for subsequent storage experiments. B cells were prepared for the luminescence assay by incubation in growth medium with the addition of 2% DMSO at a concentration of 5×10⁵ cells/mL. After 20-24 h, cells were incubated in the dark at room temperature for 2 h in assay medium [CO₂-Independent medium, 10% fetal bovine serum, 50-μg/ml streptomycin, 50-U/ml penicillin, and 250-ng/mL amphotericin B (Life Technologies)] with 50-μM coelenterazine (Molecular Probes, Eugene, Oreg.). The cells were then washed twice, resuspended in assay medium at a final concentration of 5×10⁵ cells/mL in 1.5-mL microcentrifuge tubes, and left to rotate overnight at room temperature. After preparation, cells were stored in assay medium in 1.5-mL microfuge tubes.

### Results

The addition of chemicals to the cells during preparation for the assay and/or storage was tested for improving the activity and/or viability of the cells when stored at either 4° C. or room temperature. Chemicals were chosen from the following classes: apoptosis inhibitors, antioxidants, cell-cycle inhibitors, and protease inhibitors. A full listing is given in Methods and Materials, see infra. Each additive was applied at a variety of concentrations. Viability was assessed and activity tested before storage and at 1-week intervals during storage. Those additives determined to be beneficial were then combined with one another in further experiments.

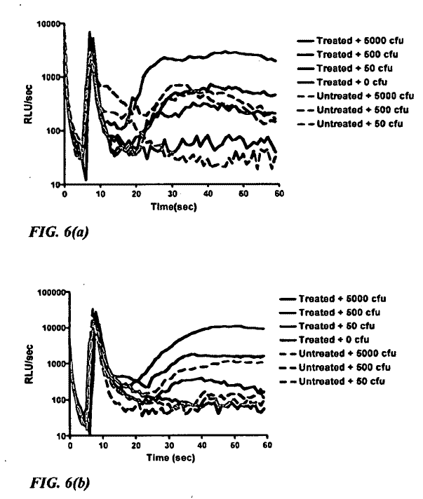

In general, treatments found to be beneficial at one storage temperature did not necessarily provide benefit at another storage temperature, and in one instance (tunicamycin treated cells stored at 4° C.) was detrimental. Improved viability and activity of cells stored at 4° C. was discovered when the cells had been incubated with wortmannin prior to preparation for the CANARY assay, and were stored in the presence of the caspase inhibitor Z-LEED-FMK and the antioxidants N-acetylcysteine, sodium selenite, deferoxamine, and/or aminoguanadine. This treatment extended the length of time the cells could be stored at 4° C., without loss of activity, from 2 weeks to 3 weeks. When applied independently, it was discovered that while wortmannin treatment improved both viability and activity, storage in Z-LEED-FMK and the antioxidants improved activity only. Similarly, it was discovered that viability and activity of cells stored at room temperature was improved when the cells had been incubated with tunicamycin prior to preparation for the assay, and were stored in the presence of Caspase Inhibitor III. This treatment extended the length of time the cells could be stored at room temperature without loss of activity from 2 days to 1 week. When applied independently, both tunicamycin treatment and storage in Caspase Inhibitor III improved both viability and activity. Other treatments that provided improvement to the activity of the cells were vacuum-sealing, storing the cells with head space, and stimulating the cells with anti-CD40 antisera. However, the response of stored cells following these treatments was inconsistent and the results shown in FIG. 6 represent results that occurred in more than half, but not more than 75%, of the experiments.

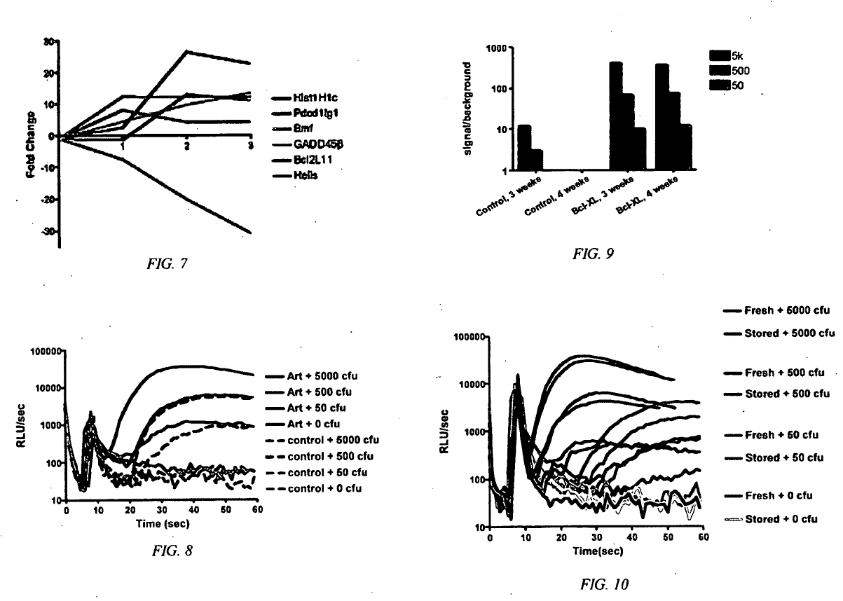

The overexpression of certain genes was also studied for a protective benefit during storage. The gene, Growth Arrest and DNA-Damage-inducible beta (GADD45β), is dramatically transcriptionally upregulated in HEK293 cells during storage, and these cells remain viable after 6 weeks of storage at room temperature [Jack et al., J. Cell Physiol. 206: 526 (2006)]. Similarly, CANARY B cells also upregulate GADD45β after preparation for the assay and maintain that level through storage (see FIG. 7). The heat-shock proteins from Artemia franciscana have been shown to confer resistance to stress when expressed in mammalian cells [see, e.g., Ma et al., Cryobiol. 51: 15 (2005); Villeneuve et al., Cell Stress Chaperones 11: 71 (2006); and Collins and Clegg, Cell Biol. Int. 28: 449 (2004)]. Overexpression of GADD45β and Artemia p26 and artemin improved the activity of the B cells stored at room temperature, increasing the storage time to 1 week without loss of activity (see FIG. 8). In general, the activity of the cells expressing artemin was better than that of cells expressing p26 or overexpressing GADD45β. Overexpression of Bcl-XL, an anti-apoptotic protein, improved both the viability and activity of cells stored at 4° C., increasing the maximum storage time from 2 to 4 weeks (see FIG. 9). In a few experiments these cells maintained full activity for up to 6 weeks. There was no benefit observed when the additives and treatments described above were applied to any of the overexpressing cell lines.

### Discussion

CANARY provides a combination of speed and sensitivity that is unmatched by other methods. However, the live-cell reagent generally has a limited shelf life of 2 days at room temperature and 2 weeks at 4° C. The reagent (i.e., CANARY cells) can be stored frozen at −80° C. or lower, but this requires liquid nitrogen or a specialized freezer. Experiments comparing stored cells to freshly prepared cells indicated that the loss of activity during storage was due not only to a decrease in viability, but also to a decrease in the amount of light emitted per cell in response to antigen (see FIG. 10). Applicants therefore sought methods to preserve both the responsiveness as well as the viability of the cells over time. Applicants discovered that both caspase inhibitors and antioxidants were beneficial, presumably because the caspase inhibitors preserved viability and the antioxidants preserved either general cell health or the oxidation state of the co-factor for aequorin, coelenterazine.

HEK293 cells can be stored at room temperature for as long as 6 weeks with good survivability, and the quiescent state is key to their survival [Jack et al., J. Cell Physiol. 206: 526 (2006)]. CANARY cells were treated with chemicals that arrest the cell cycle to induce a quiescent state. Interestingly, although beneficial effects were demonstrated when cells were treated with wortmannin and tunicamycin, it was not at concentrations that inhibited cell growth.

The heat-shock response is induced under a variety of stress conditions, and is represented by proteins, including molecular chaperones and proteases, that protect the cell from aggregation and precipitation of unfolded intermediates [Winter and Jakob, Crit. Rev. Biochem. Mol. Biol. 39: 297 (2004); Mosser et al., Mol. Cell. Biol. 17: 5317 (1997)]. Expression of the Artemia heat-shock genes p26 and artemin was discovered to preserve the activity of CANARY cells stored at room temperature.

GADD45β plays a role in both cell-cycle regulation and apoptosis. It is an anti-apoptotic protein that is induced by CD40 stimulation of B cells and mediates the suppression of Fas-induced apoptosis. It has also been described as a stress-response gene, and the expression of GADD45β is upregulated in CANARY cells after treatment with DMSO, a step that Applicants have discovered to be useful to ensure consistent activity. GADD45β is not the only gene related to DNA damage that exhibits changes in expression in CANARY cells upon treatment with DMSO. While the expression of GADD45β and Histone1 H1c (Hist1H1c) increases, the expression of helicase, lymphoid-specific (Hells) decreases (see FIG. 7). However, upregulation of GADD45β is much more dramatic in HEK293 cells, which also exhibit better viability after storage. By inducing higher expression of the gene, we were able to show improved activity of CANARY cells stored at room temperature.

Although it is not proven that apoptosis is the mechanism responsible for the loss of viable cells during storage, there are several pieces of evidence that indicate that it may be at least partly responsible. First, there are at least 2 apoptosis inhibitors that improve the viability and activity of stored cells. Second, the transcriptional analysis revealed that several apoptosis facilitators, such as Bcl2-like 11 (Bcl2l11), programmed cell death 1 ligand 1 (Pdcd1lg1), and Bcl2 modifying factor (Bmf) are upregulated (see FIG. 7). Bmf has been shown to physically bind to members of the anti-apoptotic family (Bcl2, Bcl-w, Mcl-1, and Bcl-XL) and is thought to inhibit their ability to sequester and inactivate pro-apoptotic proteins, thereby de-repressing the apoptosis pathway. Furthermore, overexpression of Bmf in mammalian cell lines induces apoptosis, an effect that can be overcome by overexpressing the anti-apoptotic proteins listed above. Applicants have demonstrated improvements in both viability and activity of cells overexpressing Bcl-XL after storage at 4° C. Interestingly, while others have shown that expression of p35 also reverses the effects of Bmf overexpression, it provided no obvious benefit when expressed in CANARY cells.

Another observation was that while many of the genes overexpressed in CANARY cells had no observable effect, overexpression of XIAP completely inhibited cellular response to antigen. This is unlikely due to an unforeseen insertional effect because it occurred when both human and murine XIAP were transfected and in all clones tested.

These results indicate that the shelf life of a living cell reagent, such as an emittor cell, can be extended. Overexpression of genes that inhibit apoptosis or act as molecular chaperones have conferred protection from cell death and perhaps from degradation of cellular components, and have extended the shelf life of CANARY cells to 1 week at room temperature and at least about 4 weeks at 4° C.

While this invention has been particularly shown and described with references to preferred embodiments thereof, it will be understood by those skilled in the art that various changes in form and details may be made therein without departing from the scope of the invention encompassed by the appended claims.

The relevant teachings of all the references, patents and patent applications cited herein are incorporated herein by reference in their entirety.

What is claimed is:

1. A method for cell preservation comprising treating a cells with a caspase inhibitor, a protease inhibitor, a proteosome inhibitor or a combination thereof.

2. The method of claim 1, wherein the cell is an emittor cell.

3. A method for cell preservation comprising increasing the expression of a quiescence regulating gene, thereby increasing the time cells can remain in a quiescent state.

4. The method of claim 3, wherein the cell is an emittor cell.

5. A method for cell preservation comprising controlling expression of a gene that regulates apoptosis.

6. The method of claim 5, wherein the gene that regulate apoptosis is an Inhibitor of Apoptosis Protein (IAP).

7. The method of claim 5, wherein the expression is up-regulation of an apoptosis inhibitor gene.

8. The method of claim 5, wherein the expression is down-regulation of an apoptosis activation gene.

9. A method for cell preservation comprising inducing quiescence.

10. The method of claim 9, wherein inducing quiescence comprises inhibiting the cell cycle in the cell.

11. The method of claim 10, wherein inhibiting the cell cycle comprises adding one or more cell cycle inhibitors to the cell.

12. The method of claim 11, wherein the one or more cell cycle inhibitors is selected from the group consisting of actinomycin D, mitomycin C, rapamycin, mevastatin, tunicamycin, wortmannin and combinations thereof.

13. An emittor cell, wherein the emittor cell is an extremophile.

14. The emittor cell of claim 13, wherein the emittor cell is a desiccation-tolerant extremophile.

15. The emittor cell of claim 14, wherein the extremophile is a chlamydomonas algae.

16. The emittor cell of claim 13, wherein the extremophile expresses a receptor for a target particle and an emittor molecule.

17. The emittor cell of claim 16, wherein the receptor is an antibody.

18. The emittor cell of claim 16, wherein the receptor is an Fc receptor.

19. The emittor cell of claim 13, wherein the emittor molecule emits a photon in response to an increase in intracellular calcium.

20. The emittor cell of claim 19, wherein the emittor molecule is aequorin.
